(19) United States
(12) Patent Application Publication
(10) Pub. No.: US 2024/0315830 A1
(43) Pub. Date: Sep. 26, 2024

(54) SYNDESMOSIS TENSIONING INSTRUMENTS, IMPLANTS, SYSTEMS, AND METHODS

(71) Applicant: Vilex in Tennessee, Inc., McMinnville, TN (US)

(72) Inventors: Brock JOHNSON, McMinnville, TN (US); Daniel J. TRIPLETT, Smithfield, UT (US)

(73) Assignee: Vilex in Tennessee, Inc., McMinnville, TN (US)

(21) Appl. No.: 18/125,626

(22) Filed: Mar. 23, 2023

(51) Int. Cl. A61F 2/08; A61F 2/46

(52) U.S. Cl. CPC A61F 2/0811; A61F 2/4618; A61F 2002/0829; A61F 2002/0858

(57) ABSTRACT

A tensioner for tensioning a syndesmosis construct may include a body, a shaft, and a slide. The body may include a first marking and the slide may include a second marking. The distal end of the shaft may movably translate relative to the body from a first proximal position closer to the body to a second distal position further away from the body, such that, when the syndesmosis construct is coupled to the slide and the distal end of the shaft is moved from the first proximal position to the second distal position, the slide may translate distally along the body from a third proximal position to a fourth distal position, and a relative position of the second marking to the first marking may indicate an amount of tension force that is applied to the syndesmosis construct.

### TECHNICAL FIELD

The present disclosure generally relates to the field of orthopedic surgery. More particularly, the present disclosure relates to the treatment of syndesmosis joints, such as a tibiofibular syndesmosis joint. However, it will also be understood that the instruments, implants, systems, and methods disclosed herein can be adapted for utilization on any bone, within any type of joint, or with any orthopedic procedure, including, but not limited to replacing an anterior cruciate ligament (ACL) of the knee, a Lisfranc repair, an acromioclavicular (AC) joint repair of the collar, etc.

### BACKGROUND

A syndesmosis joint is a slightly mobile fibrous joint in which two or more bones are joined together via connective tissues. The distal tibiofibular joint is one example of a syndesmosis joint. Injuries to the ankle syndesmosis are commonly known as “high ankle sprains” or “syndesmotic ankle sprains”. High ankle sprains are described as “high” because they are located above the ankle. High ankle sprains account for approximately 15% of all ankle sprains. However, unlike the more common “lateral ankle sprains” (which typically occur when ligaments around the ankle are injured through an inward twisting of the lower leg or foot), high ankle sprains usually occur when the lower leg or foot externally rotates or twists outwardly.

Surgical treatment of injured syndesmosis joints can include implanting a tensioning construct relative to an injured syndesmosis joint in order to compress the injured syndesmosis joint and provide additional stability during the healing process. However, ensuring a proper tension amount for an implanted tensioning construct and/or achieving a predetermined or defined tension amount for an implanted tensioning construct relative to an injured syndesmosis joint has remained elusive within currently known syndesmosis joint repair techniques. Accordingly, a need exists for improved syndesmosis joint repair instruments, devices, systems, and methods that can address these shortcomings.

### SUMMARY

The various syndesmosis joint repair instruments, implants, systems, and methods of the present disclosure have been developed in response to the present state of the art, and in particular, in response to the problems and needs in the art that have not yet been fully solved by currently available syndesmosis joint repair instruments, implants, systems, and methods.

In some embodiments, a tensioner for applying tension force to a syndesmosis construct implanted relative to a syndesmosis joint of a patient may include a body, a shaft, and a slide. The body may include a proximal end, a distal end, an intermediate portion extending between the proximal and distal ends of the body, and a first marking disposed along the intermediate portion of the body. The shaft may include a proximal end, a distal end, and a longitudinal axis. The distal end of the shaft may be translatably movable relative to the body from a first proximal position closer to the body, to a second distal position further away from the body. The slide may include a second marking disposed on the slide and a coupler configured to couple at least a portion of the syndesmosis construct to the slide. In some embodiments, when the syndesmosis construct is coupled to the slide and the distal end of the shaft is moved from the first proximal position to the second distal position, the slide may be configured to translate distally along the body from a third proximal position relative to the body, to a fourth distal position relative to the body, and a relative position of the second marking on the slide to the first marking on the body may be indicative of an amount of tension force applied to the syndesmosis construct.

In some embodiments of the tensioner, the first marking may be located on the intermediate portion of the body.

In some embodiments of the tensioner, the first marking may be located on a discrete scale coupled to the intermediate portion of the body.

In some embodiments of the tensioner, the first marking may include a plurality of first markings disposed along the intermediate portion of the body, wherein each of the plurality of first markings may be configured to indicate a different amount of tension force applied to the syndesmosis construct.

In some embodiments of the tensioner, the second marking may include a plurality of second markings disposed along the slide, wherein each of the plurality of second markings may be configured to indicate a different amount of tension force applied to the syndesmosis construct.

In some embodiments of the tensioner, the slide may also include a window formed therethrough and configured to display the first marking on the body in positional relationship to the second marking on the slide.

In some embodiments of the tensioner, the second marking disposed on the slide may include at least one edge of the slide.

In some embodiments, a tensioner for applying tension force to a syndesmosis construct implanted relative to a syndesmosis joint of a patient may include a body, a shaft, and a slide. The body may include a proximal end, a distal end, an intermediate portion extending between the proximal and distal ends of the body. The shaft may include a proximal end, a distal end, and an engagement tip located on the distal end of the shaft shaped to engage a first portion of the syndesmosis construct. The engagement tip may be translatably movable relative to the body from a first proximal position closer to the body, to a second distal position further away from the body. The slide may be configured to couple with a second portion of the syndesmosis construct and translate distally along the body in response to tension applied to the second portion of the syndesmosis construct. When the engagement tip is engaged with the first portion of the syndesmosis construct, the second portion of the syndesmosis construct is coupled to the slide, and the engagement tip is moved from the first proximal position to the second distal position: a tension force may be applied to the syndesmosis construct, and the slide may translate distally along the body from a third proximal position relative to the body to a fourth distal position relative to the body in relation to an amount of tension force applied to the syndesmosis construct.

In some embodiments of the tensioner, the engagement tip located on the distal end of the shaft may be shaped to engage a lateral button of the syndesmosis construct.

In some embodiments of the tensioner, the engagement tip may include at least one prong shaped to be received within at least one recess formed in the first portion of the syndesmosis construct.

In some embodiments of the tensioner, the engagement tip may include a first prong shaped to be received within a first recess formed in the first portion of the syndesmosis construct, and a second prong, opposite the first prong, shaped to be received within a second recess formed in the first portion of the syndesmosis construct, opposite the first recess.

In some embodiments of the tensioner, the engagement tip may include an engagement tip slot intermediate the first prong and the second prong that may be configured to receive a ridge of the first portion of the syndesmosis construct that is formed intermediate the first recess and the second recess.

In some embodiments of the tensioner, the engagement tip may include at least one angled ramp shaped to guide the second portion of the syndesmosis construct during tension.

In some embodiments of the tensioner, the engagement tip may include a first angled ramp shaped to guide a first flexible element of the syndesmosis construct during tension, and a second angled ramp shaped to guide a second flexible element of the syndesmosis construct during tension.

In some embodiments, a tensioner for applying tension force to a syndesmosis construct implanted relative to a syndesmosis joint of a patient may include a body, a shaft, and a slide. The body may include a proximal end, a distal end, and an intermediate portion extending between the proximal and distal ends of the body. The shaft may include a proximal end, a distal end, and a longitudinal axis. The distal end of the shaft may be translatably movable relative to the body from a first proximal position closer to the body, to a second distal position further away from the body. The slide may include a first coupler configured to couple a first flexible element of the syndesmosis construct to the slide, and a second coupler configured to couple a second flexible element of the syndesmosis construct to the slide. When the first and second flexible elements are coupled to the slide and the distal end of the shaft is moved from the first proximal position to the second distal position: a first tension force may be applied to the first flexible element, a second tension force may be applied to the second flexible element, and the slide may translate distally along the body from a third proximal position relative to the body to a fourth distal position relative to the body, in relation to a combined magnitude of the first and second tension forces that are applied to the first and second flexible elements.

In some embodiments of the tensioner, the slide may include a first wing projecting away from the slide along a first direction, and a second wing projecting away from the slide along a second direction.

In some embodiments of the tensioner, the first and second directions may be opposite each other.

In some embodiments of the tensioner, the first wing may include a first shoulder configured to receive at least a portion of the first flexible element thereon, and the second wing may include a second shoulder configured to receive at least a portion of the second flexible element thereon.

In some embodiments of the tensioner, the first coupler may be formed on the first wing, and the second coupler may be formed on the second wing.

In some embodiments of the tensioner, the first coupler may include a first proximal cleat configured to receive at least a first portion of the first flexible element therein, and a first distal cleat configured to receive at least a second portion of the first flexible element therein. The second coupler may also include a second proximal cleat configured to receive at least a first portion of the second flexible element therein, and a second distal cleat configured to receive at least a second portion of the second flexible element therein.

These and other features and advantages of the present disclosure will become more fully apparent from the following description and appended claims or may be learned by the practice of the systems and methods set forth hereinafter.

Exemplary embodiments of the disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the present disclosure will be described with additional specificity and detail through use of the accompanying drawings in which:

### BRIEF DESCRIPTION OF THE DRAWINGS

FIG. 1 is a perspective view of a tensioner, according to an embodiment of the present disclosure;

FIG. 2 is an exploded view of the tensioner of FIG. 1;

FIG. 3A is a side view of shaft, according to an embodiment of the present disclosure;

FIG. 3B is a top view of the shaft of FIG. 3A;

FIG. 3C is a perspective view of a distal end of the shaft of FIG. 3A;

FIG. 4A is a front perspective view of a knob, according to an embodiment of the present disclosure;

FIG. 4B is a rear perspective view of the knob of FIG. 4A;

FIG. 4C is a side view of the knob of FIG. 4A;

FIG. 4D is a cross-sectional side view of the knob of FIG. 4A, taken along the line A-A shown in FIG. 4C;

FIG. 5 is a side view of the knob of FIG. 4A assembled to the shaft of FIG. 3A;

FIG. 6 is a cross-sectional side view of the assembly shown in FIG. 5, taken along the line B-B;

FIG. 7A is a front side view of a slide, according to an embodiment of the present disclosure;

FIG. 7B is a perspective view of a distal end of the slide of FIG. 7A;

FIG. 7C is a perspective view of a proximal end of the slide of FIG. 7A;

FIG. 7D is a right side view of the slide of FIG. 7A;

FIG. 7E is a cross-sectional side view of the slide of FIG. 7A, taken along the line C-C in FIG. 7D;

FIG. 8A is a perspective view of a distal end of a body, according to an embodiment of the present disclosure;

FIG. 8B is a perspective view of a proximal end of the body shown in FIG. 8A;

FIG. 9A is a perspective view of a distal end of a body cap, according to an embodiment of the present disclosure;

FIG. 9B is a perspective view of proximal end of the body cap of FIG. 9A;

FIG. 10A is a perspective view of scale, according to an embodiment of the present disclosure;

FIG. 10B is a top view of the scale of FIG. 10A;

FIG. 10C is a bottom view of the scale of FIG. 10A;

FIG. 11 is an exploded view of a body assembly, according to an embodiment of the present disclosure;

FIG. 12 is a perspective view of the body assembly of FIG. 11, after assembly;

FIG. 13 is a perspective view of body and shaft assembly, according to an embodiment of the present disclosure;

FIG. 14 is a perspective view of an assembled tensioner at rest;

FIG. 15 is a perspective view of the tensioner of FIG. 14 in a first tension state;

FIG. 16 is a perspective view of the tensioner of FIG. 14 in a second tension state;

FIG. 17 is a perspective view of the tensioner of FIG. 14 in a third tension state;

FIG. 18 is a perspective view of suture loop construct, according to an embodiment of the present disclosure;

FIG. 19 is a perspective view of medial button, according to an embodiment of the present disclosure;

FIG. 20 is a perspective view of lateral button, according to an embodiment of the present disclosure;

FIG. 21 is a perspective view of assembled syndesmosis construct, according to an embodiment of the present disclosure;

FIG. 22 is a perspective view of an ankle syndesmosis joint;

FIG. 23 is a perspective view of the ankle syndesmosis joint of FIG. 22 with a bone plate attached to the fibula, according to an embodiment of the present disclosure;

FIG. 24 is a perspective view of the ankle syndesmosis joint of FIG. 23 with a drill bit placed through the bone plate, the fibula, and the tibia;

FIG. 25 is a lateral perspective view of the ankle syndesmosis joint of FIG. 23 with a guide rod pulling the syndesmosis construct of FIG. 21 through the ankle syndesmosis joint;

FIG. 26 is a medial perspective view of the ankle syndesmosis joint of FIG. 25 with the guide rod pulling the syndesmosis construct through the ankle syndesmosis joint;

FIG. 27 is a perspective view of the guide rod shown in FIG. 25 being threaded through the medial button shown in FIG. 19;

FIG. 28 is a perspective view of the suture loop construct attached to the medial button after the guide rod shown in FIG. 27 has been threaded through the medial button;

FIG. 29 is a perspective view of the medial button and suture loop construct of FIG. 28 engaged against the medial side of the tibia;

FIG. 30 is a lateral perspective view of the ankle syndesmosis joint of FIG. 29 with the lateral button engaged with the bone plate;

FIG. 31 is a close up perspective view of the bone plate and lateral button construct shown in FIG. 30;

FIG. 32 is a perspective view of the ankle syndesmosis joint of FIG. 30 with the distal tip of the tensioner shown in FIG. 14 engaged against the lateral button;

FIG. 33 is a perspective view of the tensioner shown in FIG. 32 with the first and second flexible elements of the syndesmosis construct coupled to the slide of the tensioner, in preparation for a syndesmosis construct tensioning procedure;

FIG. 34 is a lateral perspective view of two syndesmosis constructs fully implanted across the ankle syndesmosis joint of FIG. 32, according to an example of the present disclosure; and

FIG. 35 is a medial perspective view of the syndesmosis construct and ankle joint shown in FIG. 34.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

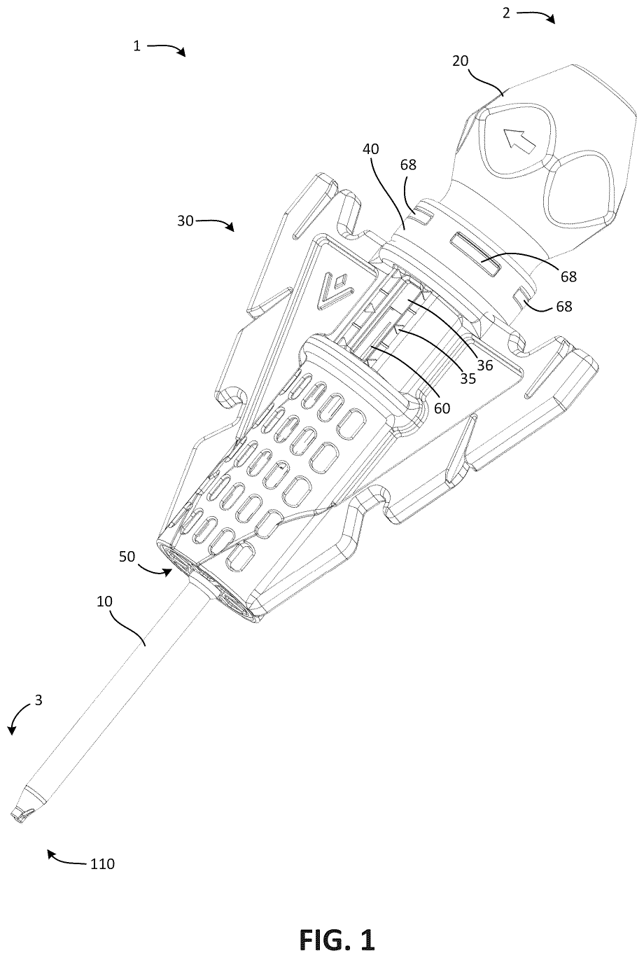

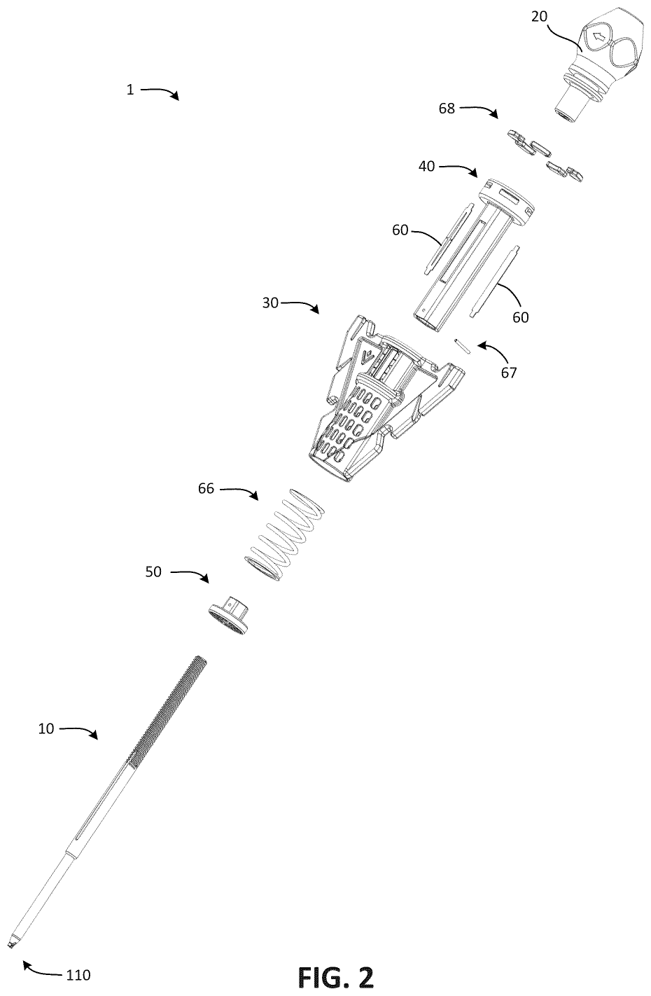

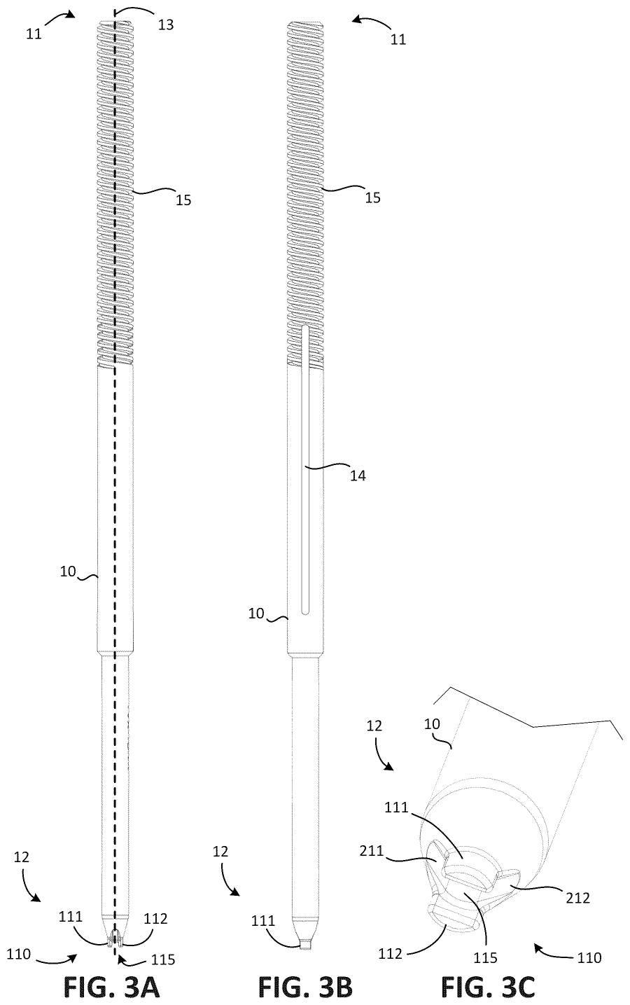

FIGS. 1-13 illustrate various views of a tensioner 1 instrument (and its component parts/subassemblies) that may be utilized to apply tension force to a syndesmosis construct, according to an embodiment of the present disclosure. Specifically, FIG. 1 is a perspective view of the tensioner 1, FIG. 2 is an exploded view of the tensioner, and FIGS. 3A-13 illustrate various views of the components and subassemblies of the tensioner 1.

Referring to FIGS. 1 and 2, in at least some embodiments the tensioner 1 may generally include a proximal end 2, a distal end 3, a shaft 10, an end cap 50, a resilient member 66, a slide 30, a body 40, a body pin 67, one or more discrete scales or scale 60, one or more knob lock tabs or lock tabs 68, and a rotational knob or knob 20.

Referring to FIGS. 3A-3C, in some embodiments the shaft 10 may generally include a proximal end 11, a distal end 12, a longitudinal axis 13, a first thread or shaft thread 15 disposed about the proximal end 11 of the shaft 10, and a shaft guide slot 14 formed through an intermediate portion of the shaft 10.

In some embodiments of the shaft 10, the distal end 12 of the shaft 10 may also include an engagement tip 110 located on the distal end 12 of the shaft 10 that may be shaped to engage at least a first portion of a syndesmosis construct, as will be discussed below in more detail with respect to FIGS. 20, 21 and 32. However, it will also be understood that some embodiments of the shaft 10 may not include the engagement tip 110 shown in FIGS. 3A-3C and/or may include different engagement tip morphologies suitable to engage any size/shape syndesmosis construct or bone plate.

Continuing with FIGS. 3A-3C, in some embodiments the engagement tip 110 may include at least one prong or first prong 111, a second prong 112 opposite the first prong 111, an intermediate slot or engagement tip slot 115 located intermediate the first prong 111 and the second prong 112, at least one angled ramp or first angled ramp 211, and a second angled ramp 212 opposite the first angled ramp 211.

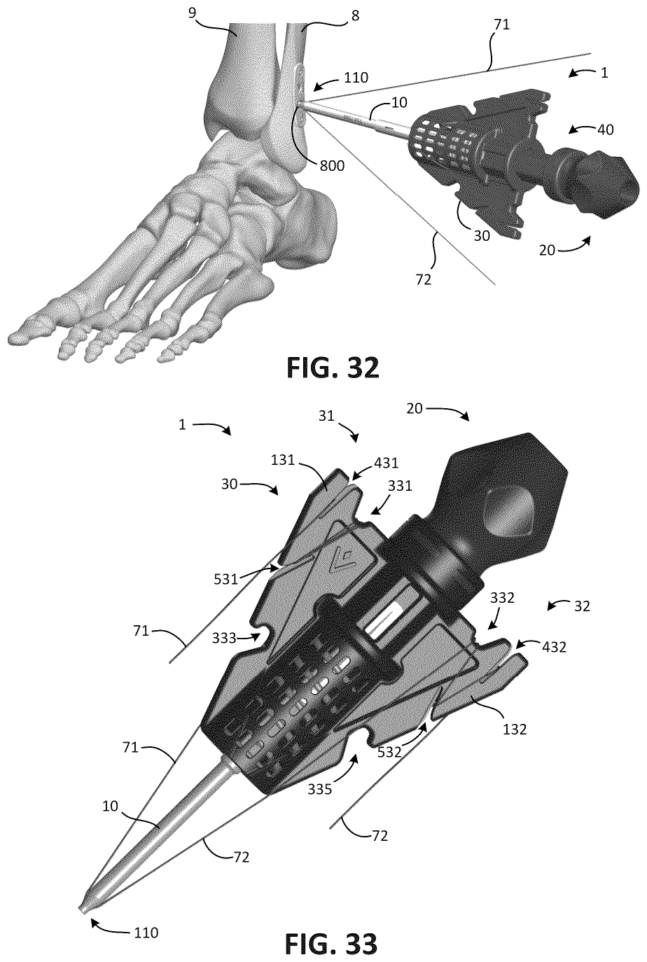

In some embodiments, the engagement tip 110 located on the distal end of the shaft 10 may be shaped to engage a lateral button 800 (e.g., see FIGS. 20 and 32) of the syndesmosis construct.

In some embodiments, the engagement tip 110 may comprise at least one prong shaped to be received within at least one recess formed in the lateral button 800.

In some embodiments, the first prong 111 of the engagement tip 110 may be shaped to be received within a first recess 801 (see FIGS. 20 and 32) formed in the lateral button 800, and the second prong 112 of the engagement tip 110 may be shaped to be received within a second recess 802 formed in the lateral button 800, opposite the first recess 801.

In some embodiments, the engagement tip 110 may include an engagement tip slot 115 intermediate the first prong 111 and the second prong 112 configured to receive a ridge 805 of the lateral button 800 that is formed intermediate the first recess 801 and the second recess 802.

In some embodiments, the first angled ramp 211 may be shaped and/or angled to guide a first flexible element 71 of the syndesmosis construct during a tensioning procedure.

In some embodiments, the second angled ramp 212 may be shaped and/or angled to guide a second flexible element 72 of the syndesmosis construct during a tensioning procedure.

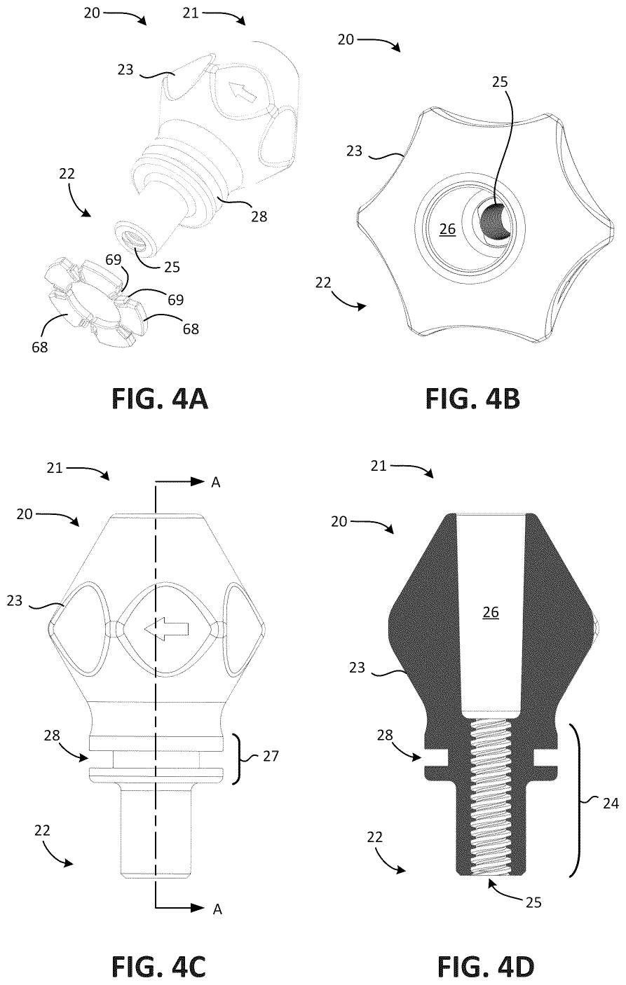

Referring to FIGS. 4A-4D, in some embodiments the knob 20 may include a proximal end 21, a distal end 22, an outer grip surface 23, a threaded knob portion 24 comprising a second thread or knob thread 25, a knob shaft passage 26, a body couple portion 27, and an annular lock tab groove 28.

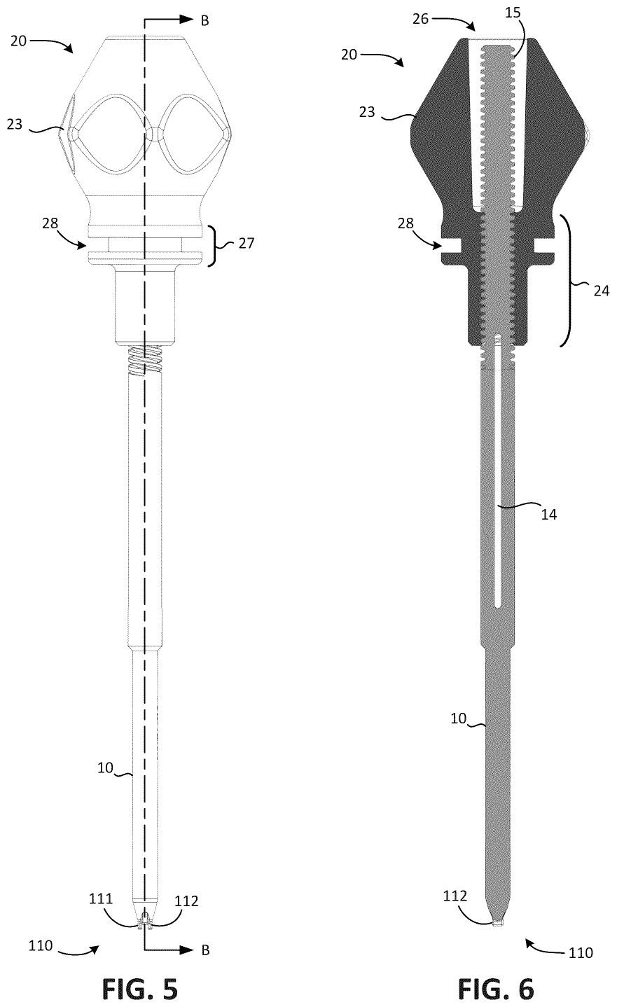

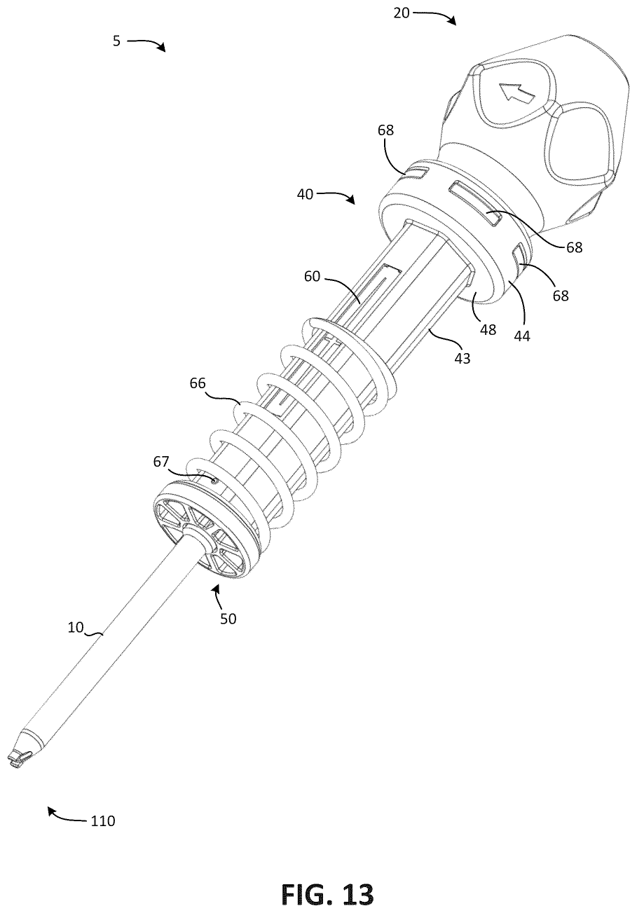

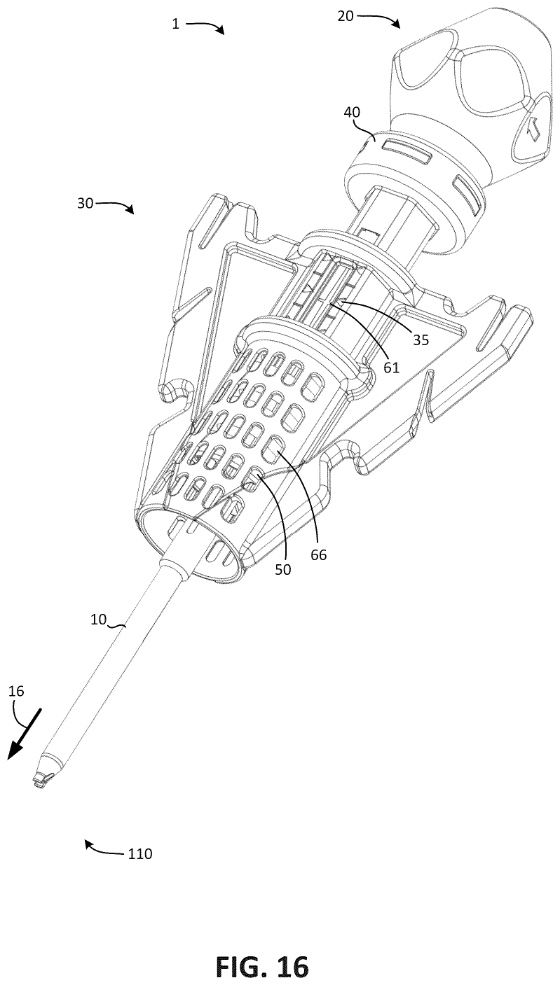

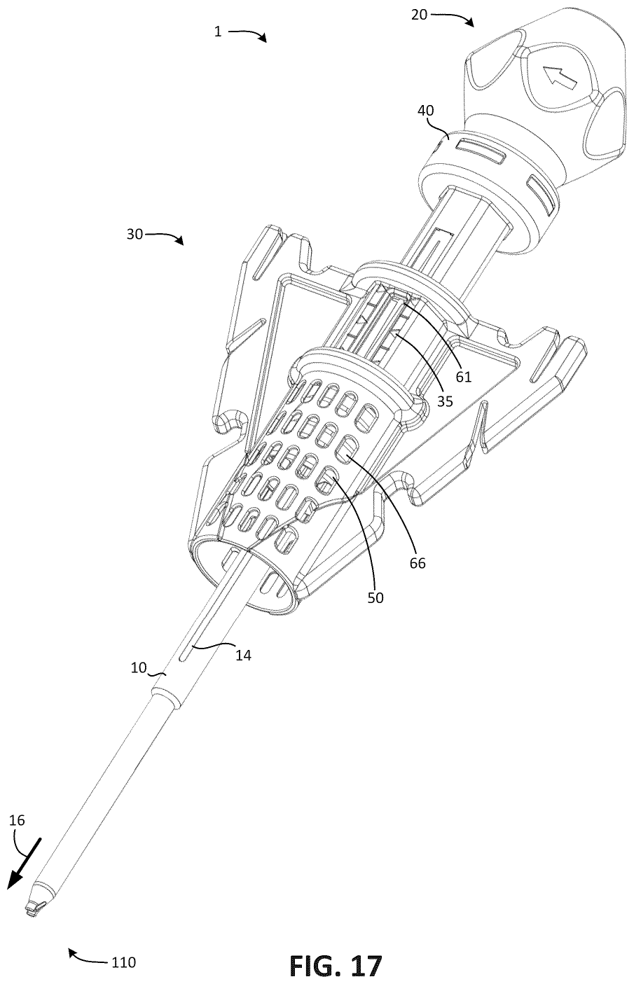

FIGS. 5 and 6 show the knob 20 and shaft 10 assembled together. Specifically, FIG. 5 is a side view of the knob 20 and shaft 10 assembly, and FIG. 6 is a cross-sectional side view of the assembly taken along the line B-B in FIG. 5. As will be discussed in more detail with respect to FIGS. 13-17 below, in some embodiments rotating the knob 20 in a first direction (e.g., clockwise) may cause the shaft 10 to translate distally with respect to the knob 20 and rotating the knob 20 in a second direction (e.g., counter-clockwise) may cause the shaft 10 to translate distally with respect to the knob 20.

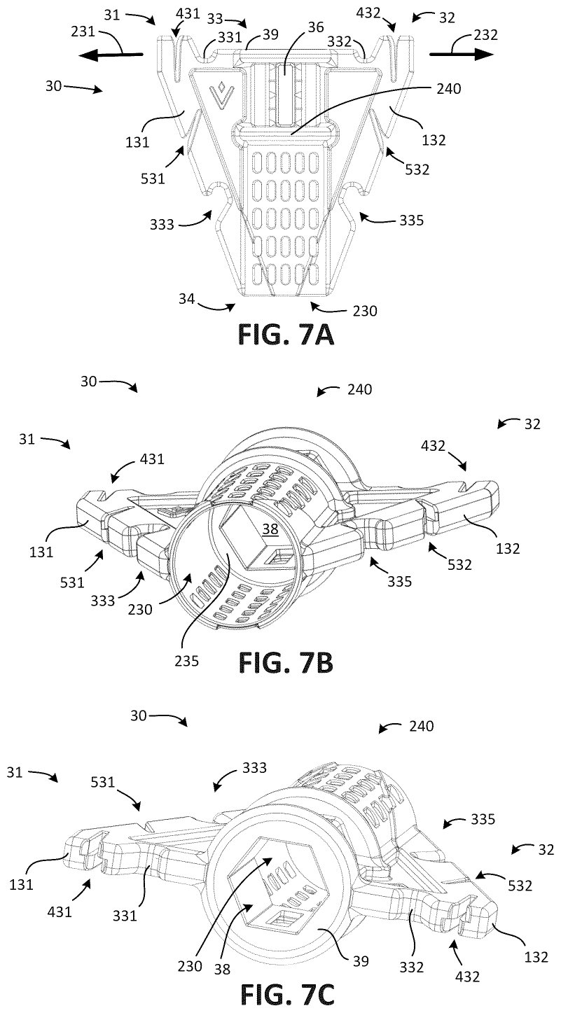

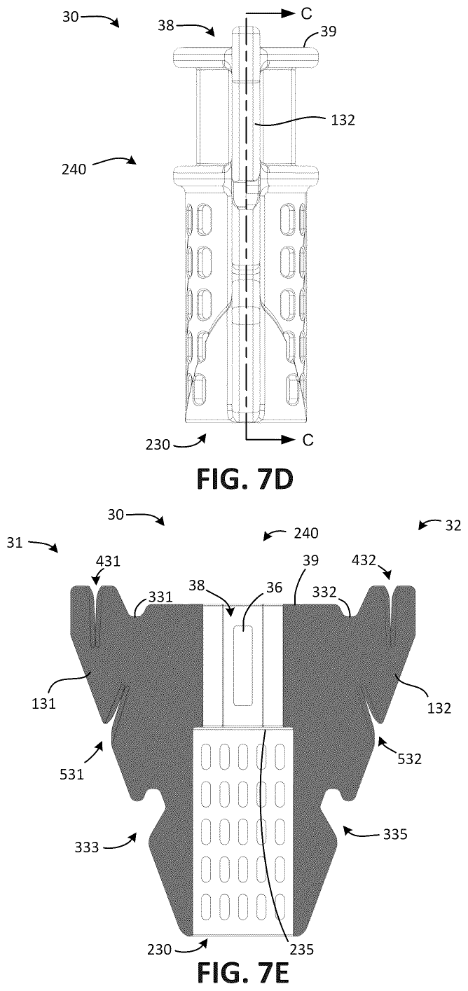

Referring to FIGS. 7A-7E, in some embodiments the slide 30 may generally include a proximal end 33, a distal end 34, a first wing 131, a second wing 132, and a slide housing 240 comprising a resilient member cavity 230 and a keyed body passage 38 formed therein.

In some embodiments, the keyed body passage 38 may be formed within the proximal end 33 of the slide housing 240, and the resilient member cavity 230 may be formed within the distal end 34 of the slide housing 240.

In some embodiments, the keyed body passage 38 may be shaped to slidingly receive at least a portion of the body 40 therein.

In some embodiments, the keyed body passage 38 may be shaped to slidingly receive at least a portion of the body 40 therein while also preventing the slide 30 from rotating with respect to the body 40 as the slide translates proximally/distally along the body 40.

In some embodiments, the keyed body passage 38 may comprise a hexagonal shape configured to slidingly receive at least a portion of the body 40 therein which may also comprise a corresponding hexagonal shape. However, it will be understood that the keyed body passage 38 and/or the body 40 may comprise any shape that allows a sliding relationship between the slide 30 and the body 40.

In some embodiments, the resilient member cavity 230 may comprise a cylindrical shape. However, it will be understood that the resilient member cavity 230 may comprise any shape that allows a sliding relationship between the slide 30 and the body 40.

In some embodiments, the keyed body passage 38 may have a smaller cross-sectional shape/diameter in comparison to a corresponding cross-sectional shape/diameter of the resilient member cavity 230.

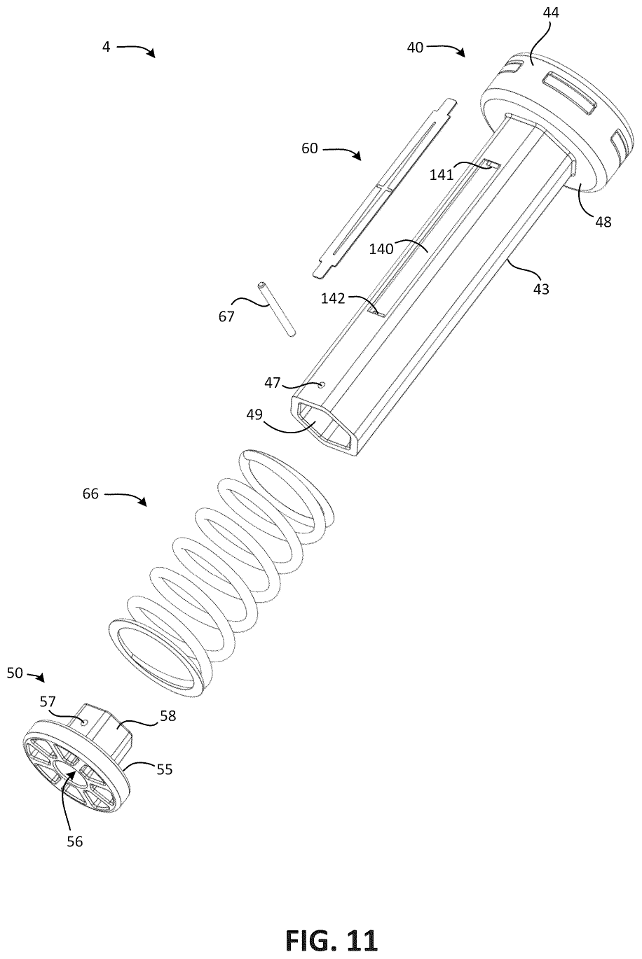

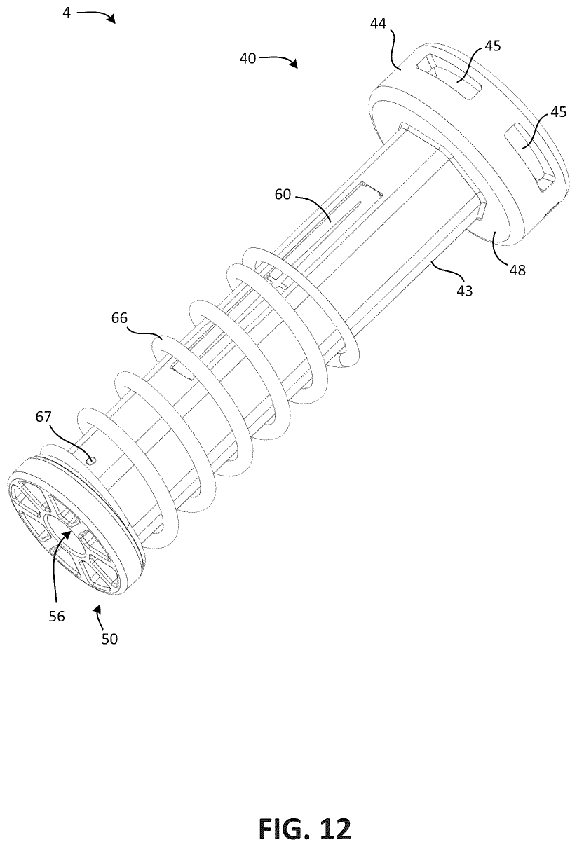

In some embodiments, an interface between the resilient member cavity 230 and the keyed body passage 38 may define a slide compression surface 235. In these embodiments, the slide compression surface 235 may engage a proximal end of the resilient member 66 such that the slide 30 compresses the resilient member 66 as the slide 30 translates distally relative to the body 40, as will be discussed in more detail below with respect to FIGS. 11-17.

In some embodiments, the slide 30 may include at least one window or window 36 formed therethrough. In these embodiments, the window 36 may be configured to display a surface of the body 40 therethrough as the slide 30 translates along the body 40.

In some embodiments, the window 36 may have a second marking 35 (or a plurality of second markings) located adjacent the window 36 that may be configured to indicate an amount of tension force that is applied to a syndesmosis construct by the tensioner via a translational position of the second marking 35 relative to the body 40.

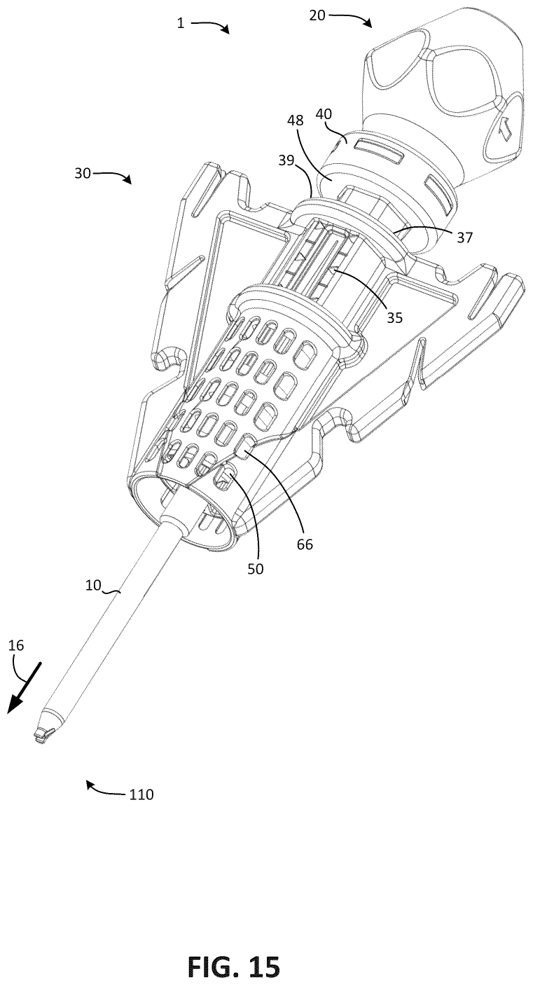

In some embodiments, at least one edge of the slide 30 itself may be configured to indicate an amount of tension force applied to a syndesmosis construct by the tensioner via a translational position of the at least one edge of the slide 30 relative to the body 40. For example, FIG. 15 shows an edge 37 of the slide 30 that may be utilized to indicate an amount of tension force applied to a syndesmosis construct by the tensioner via its translational position along the body 40 during a tensioning procedure. In some embodiments, the edge 37 may be a slide stop surface 39 located at a proximal end 33 of the slide housing 240. However, it will be understood that any edge, structure, or feature of the slide 30 may be utilized to indicate an amount of tension force applied to a syndesmosis construct via its translational position along the body 40 during a tensioning procedure.

In some embodiments, the first wing 131 may project away from the slide 30 (or slide housing 240) along a first direction 231.

In some embodiments, the second wing 132 may project away from the slide 30 (or slide housing 240) along a second direction 232.

In some embodiments, the first direction 231 and the second direction 232 may be opposite each other. However, it will also be understood that the slide 30 may include any number of wings projecting away from the slide 30 or slide housing 240 along any number of different directions (or no wings at all). Moreover, any wing may comprise any height, width, depth/thickness, shape, etc., without departing from the spirit or scope of the present disclosure.

In some embodiments, the slide 30 may include at least one coupler. The at least one coupler may be configured to removably couple at least a portion of a syndesmosis construct (e.g., such as a flexible portion of the syndesmosis construct) to the slide 30 to facilitate a tensioning procedure.

In some embodiments, the slide 30 may include a first coupler 31 and a second coupler 32.

In some embodiments, the first coupler 31 may be configured to removably couple with at least a portion of a first flexible element of the syndesmosis construct, and the second coupler 32 may be configured to removably couple with at least a portion of a second flexible element of the syndesmosis construct.

In some embodiments, the first coupler 31 and the second coupler 32 may be coupled to or formed on the slide 30 or slide housing 240.

In some embodiments, the first coupler 31 and the second coupler 32 may be coupled to or formed on the slide 30 or slide housing 240 on opposing sides of the slide 30 or slide housing 240. However, it will be understood that the first coupler 31 and the second coupler 32 may be coupled to or formed on the slide 30 or slide housing 240 on any side of the slide 30 or slide housing 240 relative to each other.

In some embodiments, the first coupler 31 may be formed on the first wing 131 and the second coupler 32 may be formed on the second wing 132.

In some embodiments, the first coupler 31 may comprise one or more features that are coupled to or formed in the first wing 131 including, but not limited to: a first proximal shoulder or first shoulder 331, a first distal shoulder 333, a first proximal cleat 431, and/or a first distal cleat 531.

In some embodiments, the second coupler 32 may comprise one or more features that are coupled to or formed in the second wing 132 including, but not limited to: a second proximal shoulder or second shoulder 332, a second distal shoulder 335, a second proximal cleat 432, and/or a second distal cleat 532.

With reference to FIG. 33, in some embodiments the first coupler 31 may be configured to couple the first flexible element 71 of the syndesmosis construct to the first wing 131 of the slide 30 by engaging and/or wrapping the first flexible element 71 around at least one of: the first shoulder 331, the first proximal cleat 431, the first distal cleat 531, and/or the first distal shoulder 333 (as desired) in order to securely couple the first flexible element 71 to the first wing 131.

Likewise, in some embodiments the second coupler 32 may be configured to couple the second flexible element 72 of the syndesmosis construct to the second wing 132 of the slide 30 by engaging and/or wrapping the second flexible element 72 around at least one of: the second shoulder 332, the second proximal cleat 432, the second distal cleat 532, and/or the second distal shoulder 335 (as desired) in order to securely couple the second flexible element 72 to the second wing 132.

In some embodiments, the cleats may comprise narrowing slits formed in the wings that are configured to wedge/trap the flexible elements therein and securely couple them to the wings during a tensioning procedure.

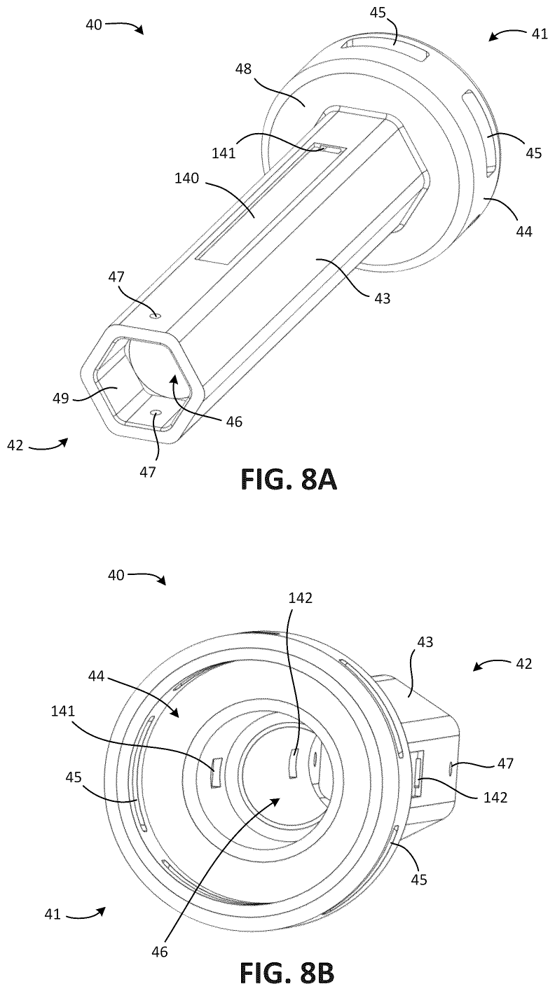

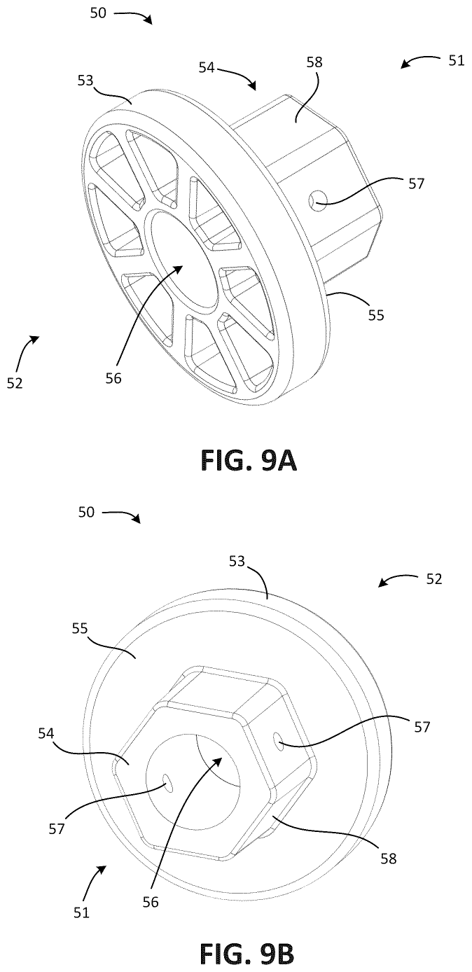

Referring to FIGS. 8A-9B, in some embodiments the body 40 may generally include a proximal end 41, a distal end 42, an intermediate portion 43 with a body shaft passage 46 formed therethrough, a knob couple portion 44 at the proximal end 41 of the body 40, and a keyed inner body surface 49 formed in the distal end 42 of the body 40. In some embodiments, the end cap 50 may generally include a proximal end 51, a distal end 52, an end cap stop 53 on the distal end 52 of the end cap 50 with an end cap compression surface 55, and an end cap body coupler 54 on the proximal end 51 of the end cap 50. The end cap body coupler 54 may include a shaft passage 56 and end cap pin holes 57 formed therethrough, as well as a keyed end cap outer surface 58.

With reference to FIGS. 8A and 8B, in some embodiments the intermediate portion 43 of the body 40 may comprise a hexagonal shape configured to be slidingly received within the hexagonally shaped keyed body passage 38 of the slide 30. As previously discussed, this configuration may also prevent the slide 30 from rotating with respect to the body 40 as the slide translates proximally/distally along the intermediate portion 43 of the body 40. However, it will be understood that the intermediate portion 43 of the body 40 and/or the keyed body passage 38 of the slide 30 may comprise any shape that allows a sliding relationship between the slide 30 and the body 40.

In some embodiments, the keyed inner body surface 49 formed in the distal end 42 of the body 40 may also comprise a hexagonal shape that may be configured to receive a portion of the end cap 50 therein (see FIGS. 9A and 9B). However, it will be understood that the keyed inner body surface 49 (and/or a correspondingly shaped end cap body coupler 54 of the end cap 50) may each comprise any shape that allows a coupling relationship between the end cap 50 and the body 40. In this manner, the end cap 50 may be coupled to the distal end 42 of the body 40 by inserting the end cap body coupler 54 into the distal end 42 of the body 40, and then placing the body pin 67 through the body pin holes 47 and the end cap pin holes 57 in order to secure the end cap 50 to the distal end 42 of the body 40 (e.g., see FIGS. 11 and 12).

Continuing with FIGS. 8A and 8B, in some embodiments the knob couple portion 44 of the body 40 may be shaped to receive the body couple portion 27 of the knob 20 (see FIGS. 4A-6) therein to rotatably couple the knob 20 to the body 40 via the lock tabs 68 shown in FIG. 4A. Once the body couple portion 27 of the knob 20 has been inserted into the knob couple portion 44 of the body 40, the lock tabs 68 may then be inserted into the lock tab slots 45 formed in the knob couple portion 44 to project within the annular lock tab groove 28 formed in the knob 20. In this manner, the knob 20 may be rotationally coupled the body 40. Once the lock tabs 68 are inserted into the lock tab slots 45, the one or more resilient fingers or resilient fingers 69 (e.g., see FIG. 4A) that are coupled to each of the lock tabs 68 may then expand and prevent the lock tabs 68 from backing out of the lock tab slots 45 to securely couple the knob 20 to the body 40 (e.g., see FIG. 13).

In some embodiments, the knob couple portion 44 and/or the body couple portion 27 may each comprise generally cylindrical shapes to allow for rotational coupling of the knob 20 to the body 40. However, the knob couple portion 44 and/or the body couple portion 27 may each comprise any shape that may allow for rotational coupling between the knob 20 to the body 40.

In some embodiments, the distal end of the knob couple portion 44 may also include a body stop surface 48 that may be configured to engage the slide stop surface 39 of the slide 30 to retain the slide 30 to the body 40 after the tensioner 1 has been assembled.

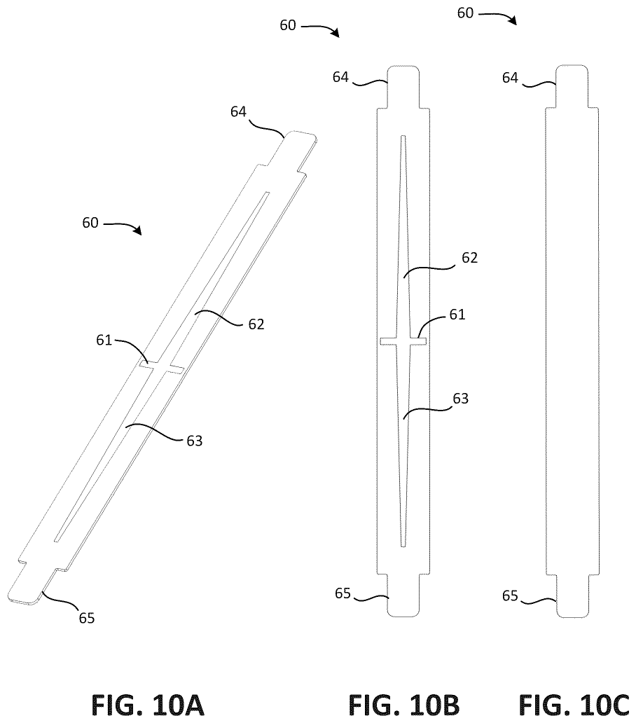

In some embodiments, the intermediate portion 43 of the body 40 may also include one or more discrete scale slots or scale slots 140 formed therein. The scale slots 140 may be configured to receive one or more discrete scales or scale 60 therein (e.g., see FIGS. 10A-10C).

In some embodiments, the scale slots 140 may include a first scale tab slot 141 and/or a second scale tab slot 142 configured to receive a first scale tab 64 and/or a second scale tab 65 of a scale 60 therein in order to secure the scale 60 to the body (e.g., see FIGS. 11, and 12).

With reference to FIGS. 10A-10C, in some embodiments the scale 60 may include a first marking 61 or a plurality of first markings.

In some embodiments, the scale 60 may include a first range marking 62 located above the first marking 61 and/or a second range marking 63 located below the first marking 61.

However, it will also be understood that in some embodiments the first marking 61 (or plurality of first markings), the first range marking 62, and/or the second range marking 63 may each be directly coupled to or formed on/in the intermediate portion 43 of the body 40 itself instead of being included on a separate scale that couples to the intermediate portion 43 of the body 40.

In some embodiments, the window 36 formed in the slide 30 may be configured to display the first marking 61 (or plurality of first markings), the first range marking 62, and/or the second range marking 63 therethrough, as previously discussed.

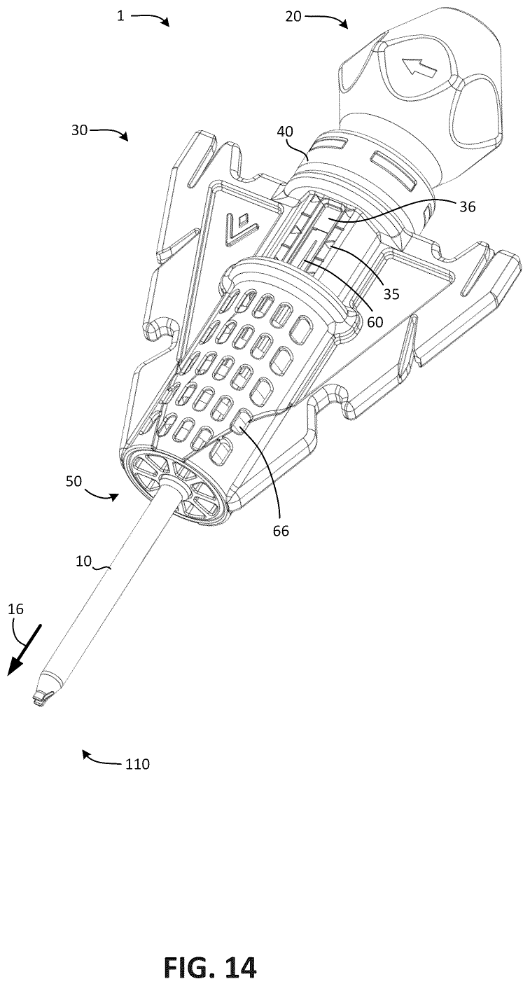

In some embodiments, a relative position of the first marking 61 (or plurality of first markings) disposed along the intermediate portion 43 of the body 40 to the second marking 35 (or plurality of second markings) disposed on the slide 30 may be configured to indicate an amount of tension force (or a plurality of different tension force magnitudes/values/ranges, etc.) applied to a syndesmosis construct during a tensioning procedure, as will be discussed in more detail below with respect to FIGS. 14-17.

FIGS. 11-14 illustrate various subassemblies for the tensioner 1, according to embodiments of the present disclosure. Specifically, FIG. 11 is an exploded view of a body subassembly 4 of the tensioner 1; FIG. 12 shows the body subassembly 4 (after assembly); FIG. 13 further includes the shaft and knob subassembly (shown in FIGS. 5 and 6) assembled to the body subassembly 4 of FIG. 12; and FIG. 14 further includes the slide 30 assembled to the body and shaft subassembly 5 of FIG. 13.

FIGS. 14-17, 32, and 33 illustrate the general operation of the tensioner 1 during a tensioning procedure. In general, the distal end or engagement tip 110 of the shaft 10 may be engaged against a first portion of a syndesmosis construct (e.g., see the engagement tip 110 engaged against the lateral button 800 in FIG. 32). Then, with the tensioner 1 in a first or resting position (e.g., see FIG. 14 showing the tensioner 1 in a resting position), the first flexible element 71 and the second flexible element 72 of the syndesmosis construct may be provisionally pulled taught by hand (see FIG. 32) at the same time by a surgeon and then respectively wrapped around the first and second wings of the tensioner 1 (see FIG. 33). At this point, the first and second flexible elements are provisionally tensioned and prepared for a final tensioning procedure by the tensioner 1.

The surgeon may then grasp the knob couple portion 44 of the body 40 with one hand and the knob 20 with the other hand. The surgeon may then rotate the knob 20 clockwise to translatably move the shaft 10 distally with respect to the body 40, causing the shaft 10 to protrude even further from the body 40 as it moves further distally (i.e., the shaft 10 effectively elongates in the distal direction 16 relative to the body 40). It will also be noted that the shaft 10 may not rotate relative to the body 40 during this distal translation process because the body pin 67 placed through the body pin holes 47, the end cap pin holes 57, and the shaft guide slot 14 may act to prevent rotation of the shaft 10 as it moves distally. Moreover, the slide 30 may also not rotate relative to the body 40 due to the hexagonal interface that exists between the slide 30 and the body 40.

FIGS. 14-17 illustrate the tensioner 1 moving from an at rest position (see FIG. 14) to various positions with increasing tension forces (see FIGS. 15-17). Note how the distal end of the shaft 10 (or engagement tip 110) moves further along the distal direction 16 as the knob 20 is further rotated in the clock-wise direction in FIGS. 14-17. Also note how the slide 30 translates further distally with respect to the body 40 as the shaft 10 translates further distally. This is so because the first flexible element 71 and the second flexible element 72 that have been provisionally tensioned and coupled to the wings (not shown in FIGS. 14-17, see FIG. 33) will act to pull the slide 30 distally via tension forces generated in the first and second flexible elements as the distal end of the shaft 10 moves distally when the knob 20 is rotated. In this manner, the slide 30 will move distally along the body 40 in proportion to the amount of tension force that is applied to the first and second flexible elements.

As previously discussed, the slide 30 is configured to translate distally along the body 40. However, the resilient member 66 (see FIG. 12) is also configured to resist distal translation of the slide 30 along the body 40 because the resilient member 66 is compressed between the slide compression surface 235 and the end cap compression surface 55 as the slide translates distally along the body 40. Thus, as the slide 30 is pulled distally by the first and second flexible elements when the shaft 10 moves distally, the resilient member 66 will become more and more compressed, resulting in greater and greater tension forces that are generated within the first and second flexible elements. As previously noted, the amount of tension force generated within the flexible elements may be measured by the relative position of the first marking 61 on the body 40 to the second marking 35 on the slide 30, as the slide 30 is move distally with respect to the body 40 to different positions.

In some embodiments, the distal end of the shaft 10 may be translatably movable relative to the body 40 from a first proximal position closer to the body 40, to a second distal position further away from the body 40.

In some embodiments, when one or more flexible elements of the syndesmosis construct are coupled to the slide 30, and the distal end of the shaft 10 is moved from a first proximal position to the second distal position, the slide 30 may translate distally along the body 40 from a third proximal position relative to the body 40 to a fourth distal position relative to the body 40, and a relative position of the second marking 35 on the slide 30 to the first marking 61 on the body 40 may indicate an amount of tension force applied to the one or more flexible elements of the syndesmosis construct.

In some embodiments, the first marking 61 may comprise a plurality of first markings disposed along the intermediate portion 43 of the body 40, and each of the plurality of first markings may be configured to indicate a different amount of tension force applied to the one or more flexible elements of the syndesmosis construct.

In some embodiments, the second marking 35 may comprise a plurality of second markings disposed along the slide 30, and each of the plurality of second markings may be configured to indicate a different amount of tension force applied to the one or more flexible elements of the syndesmosis construct.

In some embodiments, the slide 30 may include a window 36 formed therethrough and configured to display the first marking 61 on the body 40 in positional relationship to the second marking 35 on the slide 30.

In some embodiments, the second marking 35 disposed on the slide 30 may comprise at least one edge, structure, or feature of the slide 30.

In some embodiments, the shaft 10 may comprise an engagement tip 110 located on the distal end of the shaft 10 and shaped to engage a first portion of the syndesmosis construct, such as a lateral button 800. The engagement tip 110 may be translatably movable relative to the body 40 from a first proximal position closer to the body 40, to a second distal position further away from the body 40. The slide 30 may be configured to couple with a second portion of the syndesmosis construct (e.g., one or more flexible elements) and translate distally along the body 40 in response to tension applied to the second portion of the syndesmosis construct. In some embodiments, when the engagement tip 110 is engaged with the first portion of the syndesmosis construct, the second portion of the syndesmosis construct is coupled to the slide 30, and the engagement tip 110 is moved from the first proximal position to the second distal position, a tension force may be applied to the syndesmosis construct and the slide 30 may translate distally along the body 40 from a third proximal position relative to the body 40 to a fourth distal position relative to the body 40 in relation to an amount of tension force applied to the syndesmosis construct.

In some embodiments, the distal end of the shaft 10 may be translatably movable relative to the body 40 from a first proximal position closer to the body 40, to a second distal position further away from the body 40. In some embodiments, when the first and second flexible elements are coupled to the slide 30, and the distal end of the shaft 10 is moved from the first proximal position to the second distal position: a first tension force may be applied to the first flexible element 71; a second tension force may be applied to the second flexible element 72; and the slide 30 may translate distally along the body 40 from a third proximal position relative to the body 40, to a fourth distal position relative to the body 40, in relation to a combined magnitude of the first and second tension forces that are applied to the first and second flexible elements.

In some embodiments, the engagement tip 110 may engage the lateral button 800 and impart a distally directed force on the lateral button 800 that is equal and opposite to the tension forces generated in the first and second flexible elements.

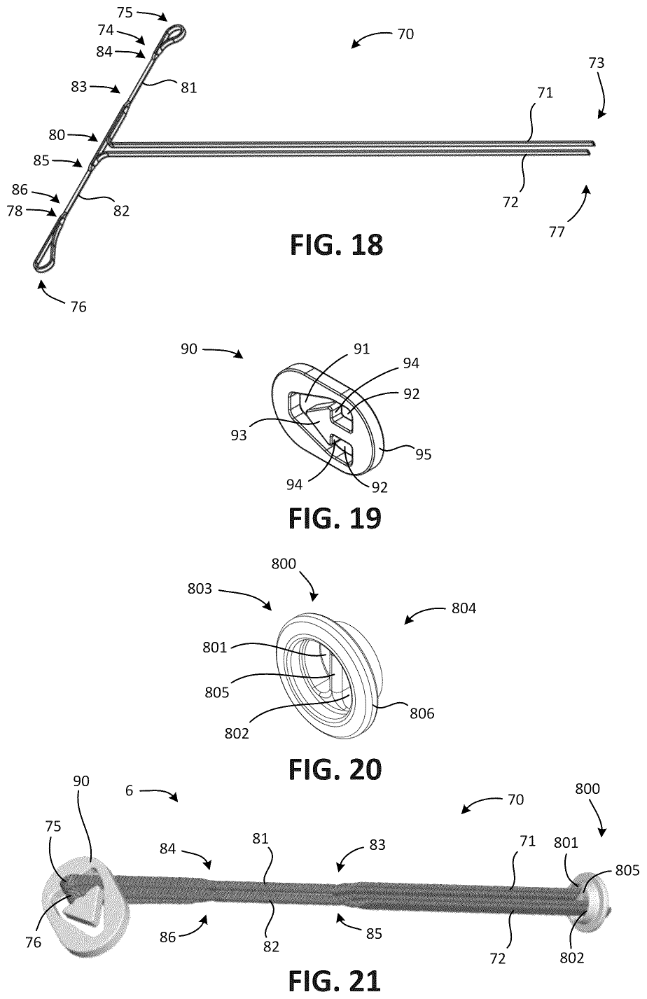

FIGS. 18-21 illustrate various components of a syndesmosis construct 6, according to an example of the present disclosure. Specifically, FIG. 18 shows a suture loop construct 70, FIG. 19 shows a medial button 90, and FIG. 20 shows a lateral button 800 of the syndesmosis construct 6 assembled together in FIG. 21.

Referring to FIG. 18, the suture loop construct 70 may generally include a first flexible element 71, a second flexible element 72, a first flexible element trap 81, a second flexible element trap 82, an intermediate suture portion 80, a first flexible element loop 75, and a second flexible element loop 76.

The first flexible element 71 may include a proximal end 73 and a distal end 74, the second flexible element 72 may include a proximal end 77 and a distal end 78, the first flexible element trap 81 may include a proximal end 83 and a distal end 84, and the second flexible element trap 82 may include a proximal end 85 and a distal end 86.

In some embodiments, the distal end 74 of the first flexible element 71 may be coupled with the distal end 84 of the first flexible element trap 81, and the proximal end 73 of the first flexible element 71 may be threaded through the first flexible element trap 81 to form the first flexible element loop 75.

In some embodiments, the distal end 78 of the second flexible element 72 may be coupled with the distal end 86 of the second flexible element trap 82, and the proximal end 77 of the second flexible element 72 may be threaded through the second flexible element trap 82 to form the second flexible element loop 76.

In some embodiments, the first flexible element trap 81 and the second flexible element trap 82 may function similar to “Chinese finger-traps” by allowing the first and second flexible elements to be pulled through the first and second flexible element traps respectively in order to tighten the first and second flexible element loops, while resisting any loosening of the first and second flexible elements through the first and second flexible element traps in the opposite direction. In this manner, the suture loop construct 70 forms a knotless suture loop construct that improves convenience and efficiency for the surgeon during a surgical procedure.

In some embodiments, the intermediate suture portion 80 may be coupled between the proximal ends of the first and second flexible element traps.

Referring to FIG. 19, the medial button 90 may generally include a medial button outer perimeter 95 enclosing a loop retainer 93 surrounded by a guide rod passage 91 and loop passages 92. The loop retainer 93 may also include loop stop surfaces 94.

Referring to FIG. 20, the lateral button 800 may generally include a lateral button outer perimeter 806 enclosing a ridge 805 located intermediate a first recess 801 formed through the lateral button 800 and a second recess 802 formed through the lateral button 800, opposite the first recess 801.

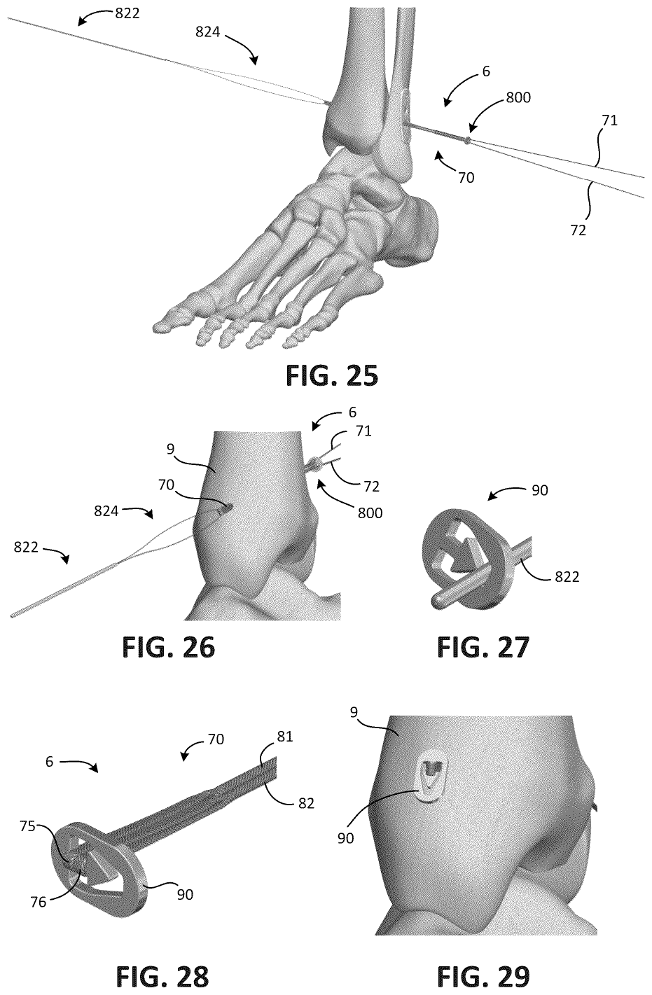

In some embodiments, the syndesmosis construct 6 may be assembled by inserting the first flexible element loop 75 through the first recess 801 of the lateral button 800 and inserting the second flexible element loop 76 through the second recess 802 of the lateral button 800. In this manner, the intermediate suture portion 80 will lie across the ridge 805 with the first and second flexible element loops projecting from a distal end 804 of the lateral button 800 and the proximal ends of the first and second flexible elements projecting from a proximal end 803 of the lateral button 800 (e.g., see FIGS. 25 and 26). The first and second flexible element loops may then be partially inserted through the guide rod passage 91 of the medial button 90 to allow the first and second flexible element loops to slide over the loop retainer 93 for retention by the loop stop surfaces 94 (e.g., see FIG. 21).

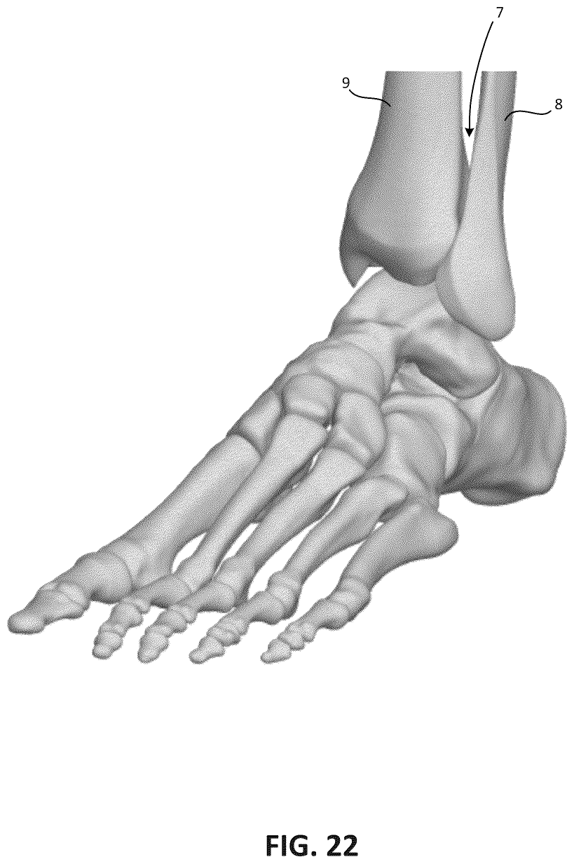

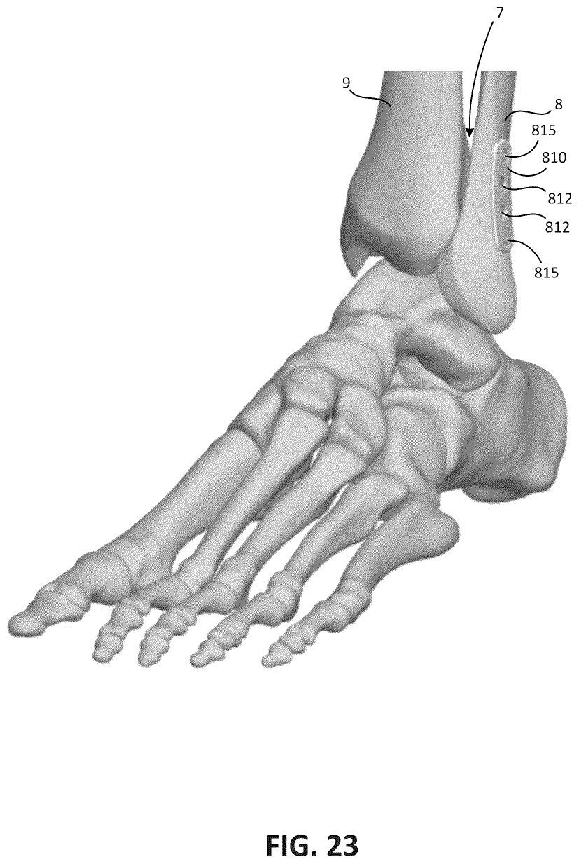

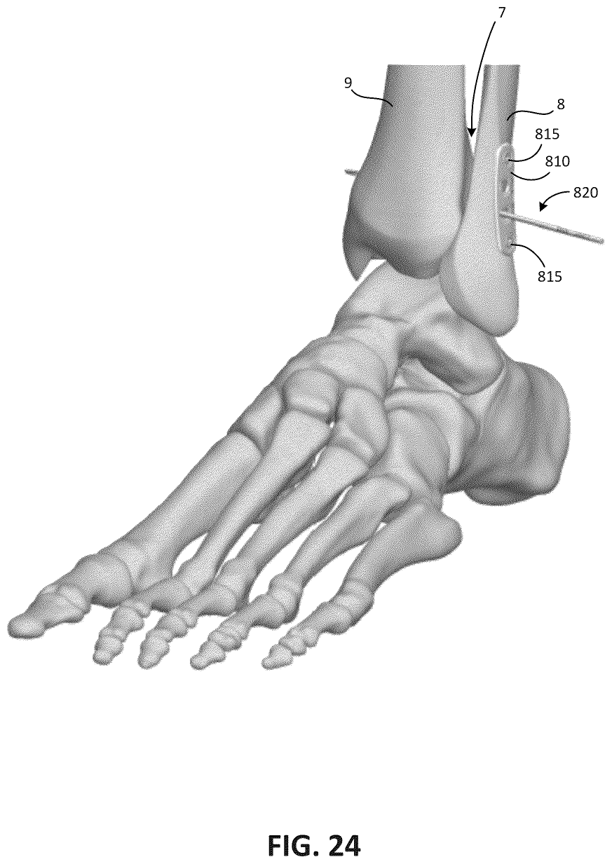

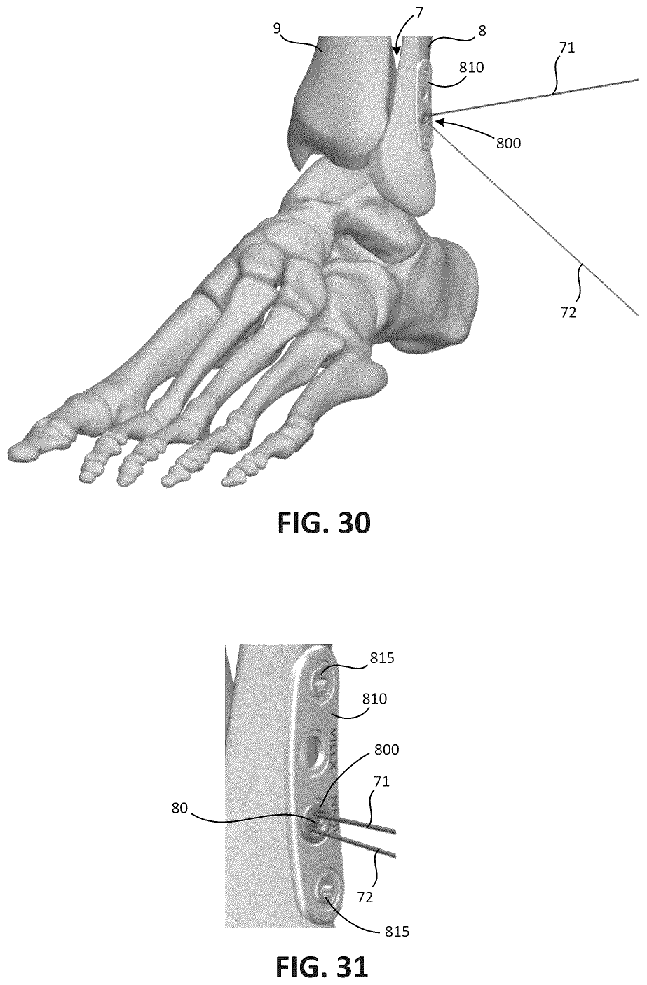

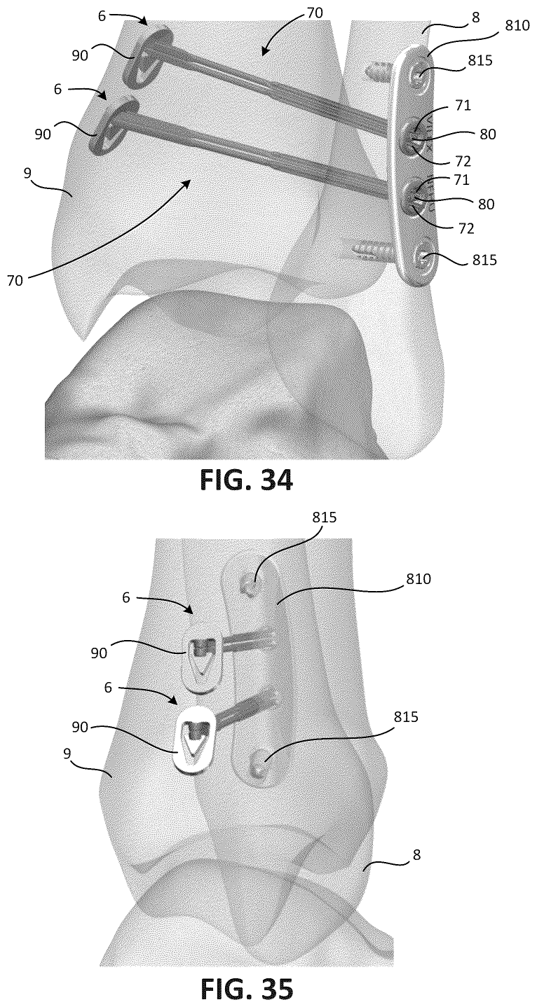

FIGS. 22-35 illustrate an example method for implanting and tensioning the syndesmosis construct 6 of FIG. 21 relative to a syndesmosis joint, such as an ankle syndesmosis joint 7 formed between a fibula 8 and a tibia 9 (see FIG. 22). In a first step of the procedure, the fibula 8 may be surgically exposed and a surface of the fibula 8 may be prepared (e.g., rasped, resurfaced, etc.) in order to receive a bone plate 810 thereon (see FIG. 23). The bone plate 810 may include a bone plate hole 812 or one or more bone plate holes that may be configured to receive a bone screw 815 or one or more bone screws and/or a syndesmosis construct 6 or one or more syndesmosis constructs therethrough. Once the bone plate 815 has been placed on the fibula 8, a drill bit 820 (see FIG. 24) may be utilized to drill a bone tunnel through the bone plate hole 812 and through the fibula 8 and the tibia 9 to receive the syndesmosis construct 6 through the bone tunnel. The drill bit 820 may then be removed and the syndesmosis construct 6 may be threaded through the bone tunnel (see FIGS. 25 and 26). A guide rod 822 and pull-through suture 824 may be utilized to pull the syndesmosis construct 6 through the bone tunnel. In some embodiments, the syndesmosis construct 6 may be fully assembled (except for the medial button 90) and/or pre-loaded onto the guide rod 822 and pull-through suture 824 for convenience. A small incision on the medial side of the tibia 9 may allow the guide rod 822 to exit through the medial side of the tibia 9. The location of the small incision on the medial side of the tibia 9 may be determined by pushing the guide rod 822 and viewing/feeling where the guide rod 822 wants to exit on the medial side of the tibia 9. Once the small incision has been made on the medial side of the tibia 9, the syndesmosis construct 6 may be pulled through the bone tunnel until the lateral button 800 seats within the bone plate hole 812 (e.g., see FIGS. 30 and 31). Moreover, the tip of the guide rod 822 may also be threaded through the guide rod passage 91 of the medial button 90 (see FIG. 27) and the first and second flexible element loops of the syndesmosis construct 6 may then be partially inserted through the guide rod passage 91 of the medial button 90 to allow the first and second flexible element loops to slide over the loop retainer 93 for retention by the loop stop surfaces 94 of the medial button 90 (see FIG. 28). The pull-through suture 824 may also be cut to release the first and second flexible element loops. The first and second flexible elements protruding from the lateral button 800 may then be provisionally tightened by hand to seat the medial button 90 against the medial side of the tibia 9 (see FIG. 29) and/or to seat the lateral button 800 against the bone plate 810 (see FIGS. 30-32). The syndesmosis construct 6 will maintain this provisional tightening due to the “Chinese finger-trap” functionality previously discussed. The engagement tip 110 of the tensioner 1 may then be engaged against the lateral button 800 (see FIG. 32) and the first and second flexible elements may be coupled to the first and second wings of the tensioner 1 (see FIG. 35) in their provisionally tightened state. The tensioner 1 may then be utilized to tension the syndesmosis construct 6 to a desired tension level/amount/range by rotating the knob 20, as previously discussed herein. Once a desired tension level/amount/range has been achieved by viewing the various markings on the tensioner 1, as previously described herein, the excess first and second flexible element portions may be trimmed close to the lateral button 800 (see FIGS. 34 and 35) and the tensioner 1 may be removed from the lateral button 800. FIGS. 34 and 35 also show how multiple syndesmosis constructs may be installed and tensioned in a syndesmosis joint, according to embodiments of the present disclosure.

It will be understood than any feature or group of features described or contemplated herein with respect to any instrument, implant, system, or method may be combined in any fashion with any other instrument, implant, system, or method that is described or contemplated herein in order to make any number of different instrument, implant, system, or method configurations.

Any procedures or methods disclosed herein comprise one or more steps or actions for performing the described procedure/method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of the appended claims is not limited to the precise configuration and components disclosed herein. Various modifications, changes, and variations which will be apparent to those skilled in the art may be made in the arrangement, operation, and details of the devices, instruments, systems and methods disclosed herein.

What is claimed is:

1. A tensioner for applying tension force to a syndesmosis construct implanted relative to a syndesmosis joint of a patient, the tensioner comprising:
a body comprising:
a proximal end;
a distal end;
an intermediate portion extending between the proximal and distal ends of the body; and
a first marking disposed along the intermediate portion of the body;
a shaft comprising:
a proximal end;
a distal end; and
a longitudinal axis,
wherein the distal end of the shaft is translatably movable relative to the body from a first proximal position closer to the body, to a second distal position further away from the body; and
a slide comprising:
a second marking disposed on the slide; and
a coupler configured to couple at least a portion of the syndesmosis construct to the slide,
wherein, when the syndesmosis construct is coupled to the slide, and the distal end of the shaft is moved from the first proximal position to the second distal position:
the slide is configured to translate distally along the body from a third proximal position relative to the body, to a fourth distal position relative to the body; and
a relative position of the second marking on the slide to the first marking on the body is indicative of an amount of tension force applied to the syndesmosis construct.

2. The tensioner of claim 1, wherein the first marking is located on the intermediate portion of the body.

3. The tensioner of claim 1, wherein the first marking is located on a discrete scale coupled to the intermediate portion of the body.

4. The tensioner of claim 1, wherein:
the first marking comprises a plurality of first markings disposed along the intermediate portion of the body,
wherein each of the plurality of first markings is configured to indicate a different amount of tension force applied to the syndesmosis construct.

5. The tensioner of claim 1, wherein:
the second marking comprises a plurality of second markings disposed along the slide,
wherein each of the plurality of second markings is configured to indicate a different amount of tension force applied to the syndesmosis construct.

6. The tensioner of claim 1, wherein:
the slide further comprises a window formed therethrough and configured to display the first marking on the body in positional relationship to the second marking on the slide.

7. The tensioner of claim 1, wherein the second marking disposed on the slide comprises at least one edge on the slide.

8. A tensioner for applying tension force to a syndesmosis construct implanted relative to a syndesmosis joint of a patient, the tensioner comprising:
a body comprising:
a proximal end;
a distal end; and
an intermediate portion extending between the proximal and distal ends of the body;
a shaft comprising:
a proximal end;
a distal end; and
an engagement tip located on the distal end of the shaft shaped to engage a first portion of the syndesmosis construct,
wherein the engagement tip is translatably movable relative to the body from a first proximal position closer to the body, to a second distal position further away from the body; and
a slide configured to couple with a second portion of the syndesmosis construct and translate distally along the body in response to tension applied to the second portion of the syndesmosis construct,
wherein, when the engagement tip is engaged with the first portion of the syndesmosis construct, the second portion of the syndesmosis construct is coupled to the slide, and the engagement tip is moved from the first proximal position to the second distal position:
a tension force is applied to the syndesmosis construct; and
the slide translates distally along the body from a third proximal position relative to the body, to a fourth distal position relative to the body, in relation to an amount of tension force applied to the syndesmosis construct.

9. The tensioner of claim 8, wherein the engagement tip located on the distal end of the shaft is shaped to engage a lateral button of the syndesmosis construct.

10. The tensioner of claim 8, wherein the engagement tip comprises at least one prong shaped to be received within at least one recess formed in the first portion of the syndesmosis construct.

11. The tensioner of claim 8, wherein the engagement tip comprises:
a first prong shaped to be received within a first recess formed in the first portion of the syndesmosis construct; and
a second prong, opposite the first prong, shaped to be received within a second recess formed in the first portion of the syndesmosis construct, opposite the first recess.

12. The tensioner of claim 11, wherein the engagement tip comprises an engagement tip slot intermediate the first prong and the second prong configured to receive a ridge of the first portion of the syndesmosis construct that is formed intermediate the first recess and the second recess.

13. The tensioner of claim 8, wherein the engagement tip comprises at least one angled ramp shaped to guide the second portion of the syndesmosis construct during tension.

14. The tensioner of claim 8, wherein the engagement tip comprises:
a first angled ramp shaped to guide a first flexible element of the syndesmosis construct during tension; and
a second angled ramp shaped to guide a second flexible element of the syndesmosis construct during tension.

15. A tensioner for applying tension force to a syndesmosis construct implanted relative to a syndesmosis joint of a patient, the tensioner comprising:
a body comprising:
a proximal end;
a distal end; and
an intermediate portion extending between the proximal and distal ends of the body;
a shaft comprising:
a proximal end;
a distal end; and
a longitudinal axis,
wherein the distal end of the shaft is translatably movable relative to the body from a first proximal position closer to the body, to a second distal position further away from the body; and
a slide comprising:
a first coupler configured to couple a first flexible element of the syndesmosis construct to the slide; and
a second coupler configured to couple a second flexible element of the syndesmosis construct to the slide;
wherein, when the first and second flexible elements are coupled to the slide, and the distal end of the shaft is moved from the first proximal position to the second distal position:
a first tension force is applied to the first flexible element;
a second tension force is applied to the second flexible element; and
the slide translates distally along the body from a third proximal position relative to the body, to a fourth distal position relative to the body, in relation to a combined magnitude of the first and second tension forces that are applied to the first and second flexible elements.

16. The tensioner of claim 15, wherein the slide comprises:
a first wing projecting away from the slide along a first direction; and
a second wing projecting away from the slide along a second direction.

17. The tensioner of claim 16, wherein the first and second directions are opposite each other.

18. The tensioner of claim 16, wherein:
the first wing comprises a first shoulder configured to receive at least a portion of the first flexible element thereon; and
the second wing comprises a second shoulder configured to receive at least a portion of the second flexible element thereon.

19. The tensioner of claim 16, wherein:
the first coupler is formed on the first wing; and
the second coupler is formed on the second wing.

20. The tensioner of claim 19, wherein:
the first coupler comprises:
a first proximal cleat configured to receive at least a first portion of the first flexible element therein; and
a first distal cleat configured to receive at least a second portion of the first flexible element therein; and
the second coupler comprises:
a second proximal cleat configured to receive at least a first portion of the second flexible element therein; and
a second distal cleat configured to receive at least a second portion of the second flexible element therein.
